(19) United States
(12) Patent Application Publication
(10) Pub. No.: US 2015/0238021 A1
(43) Pub. Date: Aug. 27, 2015

(54) SUPPORT APPARATUS FOR EQUIPMENT FOR LYING OR SITTING ON

(71) Applicant: Klemens WASSERMANN, Wien (AT)

(72) Inventor: Klemens Wassermann, Wien (AT)

(21) Appl. No.: 14/427,710

(22) PCT Filed: Sep. 13, 2013
(86) PCT No.: PCT/AT2013/050182

§ 371 Date: Mar. 12, 2015

(30) Foreign Application Priority Data
Sep. 13, 2012 (AT) A 50390/2012

(51) Int. Cl. A47C 23/28; A61G 7/015; A61F 7/00; A47C 21/04; A61H 1/00; A47C 31/12; A61G 7/018

(52) U.S. Cl. CPC A47C 23/28; A47C 31/123; A47C 31/126; A61G 7/015; A61G 7/018; A47C 21/044; A47C 21/048; A61H 1/008; A61F 7/00

(57) ABSTRACT

Support apparatus (1) for lying or sitting equipment, comprising an at least partially fixed frame (2) and comprising intertwined tensioned straps (4) which define a support surface (8) and are accessible both from the upper support side and from the opposite underside of the support surface (8), wherein at least some of the straps (4) are connected to the frame (2) by means of separately adjustable tensioning devices (7), in which case the pretensioning of the straps (4) is locally adjustable and therefore the hardness of the support surface (8) is locally adaptable at any time.

### TECHNICAL FIELD

The invention relates to a support apparatus for lying or sitting equipment, comprising an at least partially fixed frame and comprising intertwined tensioned straps which define a support surface and are accessible both from the upper support side and from the opposite underside of the support surface.

Such apparatuses are especially used with beds or couches where their robustness and their higher comfort, for example as compared to pure metal or mat constructions, has proved successful.

### STATE OF THE ART

U.S. Pat. No. 3,742,527 describes a hospital bed with a mattress support which is divided into partial segments pivotable relative to each other and which comprises straps tensioned in the longitudinal and transverse directions which are accessible from the underside. The movable parts of the frame of the mattress support are adjustable by means of pneumatic drives. Due to the inelasticity of the mattress support, the use of a softer mattress is necessary between the support and the user for the sake of comfort, so that, however, the permeability of air from the underside to the support surface is strongly reduced, inter alia, and access for a massage is practically prevented.

CN 201067225 Y discloses a mattress whose core is formed by a network of intertwined palm ropes which are fixedly arranged in a perforated rectangular wood frame. The mattress is enclosed by a moisture-tight nylon layer so as to avoid deformations of the core.

CN 2779994 Y discloses a mattress with a continuous rigid wood frame which is encompassed with intertwined straps, wherein the tensioning of the straps can only be adjusted globally for the entire support surface.

DE 199 27 096 A1 further describes a mattress of a loose mesh work of longitudinal and transverse straps arranged in a stable frame. The longitudinal and/or transverse straps are connected to the frame via rolls applied at the strap ends and a joint rope mounted in appropriate rolls on the frame such that the distances between the strap ends and the frame are compensated for via the rope and are constant in total. A local adjustability of the strap tension is not provided, but the mechanism on the contrary offsets tension differences between the straps. A loose spacer and/or a spacer designated as “floating” may be connected to the strap mesh work so as to limit the distance between adjacent straps in the areas of body bulges.

DE 27 24 316 and US 2003/0131410 A1 disclose frames which are spanned with a mesh work of substantially inelastic ribbons.

None of these known support apparatuses provides the possibility of adjusting the tensioning of the straps locally, i.e. for individual straps, so that it is a disadvantage that no adaptation of the support surface to the locally modified resilience or to the required locally different tensions can be made.

AT 179048 B1 illustrates a cushion support with a fixed frame into which a plurality of tension springs is clamped. Strips extending across the breadth and length of the cushion support are arranged between the springs, wherein a part of the strips may be clamped resiliently and elastically as well as between tension springs. The resilience of the cushion support can be determined during the manufacturing and/or during the assembly of the cushion support by an appropriate selection of the springs with respect to the spring strength, the diameter or the tension.

CH 400 482 A relates to an apparatus with a bed frame into which elastic straps are tensioned. The straps are clamped into the frame, wherein the strap tension can only be changed with effort by loosening of the clamping connection, manual pretensioning of the straps and restituting of the clamping connection. Moreover, the selection of the strap material is strongly restricted with this apparatus due to the necessary stretchability of the straps and/dr of the strap material.

### SUMMARY OF THE INVENTION

It is therefore an object of the invention to provide a support apparatus of the initially mentioned kind in which the resilience of the support surface is locally adjustable such that improved comfort during use is achieved. In particular, the hardness of the support surface is intended to be adjustable such that the use of a mattress is superfluous, so that the air permeability of the strap network which is high as compared to usual mattresses may be directly beneficial for the user. Moreover, the selection of the strap material is intended not to be restricted by demands to elasticity, but is intended to be adaptable optimally to the use of a mattress in particular with respect to air permeability, slidability and hygienic requirements. Moreover, any noise development during the movement of the straps should be kept as low as possible.

The support apparatus according to the invention of the initially mentioned kind solves this object in that at least some of the straps are connected to the frame by means of separately adjustable tensioning devices such that the pretensioning of the straps is locally adjustable and hence the hardness of the support surface is locally adaptable at any time, in particular also during the use of the support apparatus and/or in the case of loaded straps. A low noise development is achieved by a very good slidability of the strap surface. A local adjustment of the pretensioning may accordingly be made in a simple manner by actuating the respective tensioning device.

With the present support apparatus it is therefore also possible to compensate for a local weakening of the strap tensioning if the weakening due to strong local loading of the support surface only relates to a part of the straps without the necessity of increasing the tensioning of the remaining straps, too. It is likewise possible to adapt the “hardness” of the support surface in regions to the physical condition of the current user, wherein, for instance, in the case of a couch a smaller hardness is applied in the hip, knee, heel and shoulder areas. In the case of a user change or in the case of modified demands the adjustments can be changed without high effort at any time and the support characteristic can be adapted anew. Last but not least, due to their arrangement between the straps and the frame it is possible for the tensioning devices to also anticipate and/or correct modifications of the frame.

The invention can be used with particular advantage for couches/beds, but it can certainly also be used for other equipment carrying persons such as, for instance, fauteuils or similar seating furniture, up to vehicle, railway, bus or aircraft seats, and also for the backrests thereof.

It is particularly advantageous for the use of a plurality of similar equipments for lying or sitting on and for an easy exchange of parts of the support apparatus according to the invention if the frame along with the straps forms a first module having an equipment for treatment which forms a second module arranged at the underside thereof, said equipment for treatment in turn being arranged on a third module formed by a substructure. Thus, the first and generally lightest module may be separated from the rest of the support apparatus, for instance, for cleaning purposes, which facilitates transportation and cleaning. Moreover, the equipment for treatment, i.e. the second module, may be exchanged on demand, especially if different equipments for treatment with different treatment functions are available, or for example for an easy replacement of damaged equipments for treatment by maintaining the first and third modules and the adjustments made there.

In an efficient and comparatively cost-efficient variant of the apparatus according to the invention the straps connected to a tensioning device are firmly connected to the frame at their end opposite to the tensioning device. This requires only a one-side adjustment of the strap pretensioning which is, however, hardly disadvantageous in areas with relatively high pretensioning since the clearance enabled by the tensioning device is small there, anyway.

If the straps are connected at both sides with a tensioning device which preferably have the same spring stiffness, shifting of the straps in the central area of the support, and thus relative to a loaded position, can at least be avoided partially since the straps yield in the direction of both ends. Moreover, in this case larger resilience altogether and/or a larger bandwidth of possible pretensioning adjustments can be achieved with otherwise equal tensioning devices.

The finest local adjustability of the strap pretensioning is achieved if one single strap is connected to each tensioning device. In this case it is in particular possible to adjust approximately smooth transitions between areas of the support surface with higher and lower pretensioning and/or hardness in that the straps in between are adjusted with appropriately graded pretensioning. If more than one strap is connected to a tensioning device, the number of straps connected to a tensioning device may, for instance, be maximally ten straps with a breadth of less than 30 mm or maximally five straps with a breadth of approximately 50 mm.

It is further advantageous if the tensioning devices comprise a sliding or rolling guide of the straps since thus it is, for instance, possible to avoid rubbing noises of the support and to ensure that the resilience of the straps is exclusively oriented along the strap course. A loading of the tensioning device which is orthogonal to the strap course and which might have damage thereof or a distortion of the hardness adjustment for the support surface as a consequence may be avoided by the sliding or rolling guide of the straps which is operably arranged between the support surface and the tensioning device.

Moreover, in order to keep an edge region of the support surface—which is uncomfortable since it is not formed by straps—as small as possible it is favorable if the sliding and rolling guide deflects the straps in the direction of the underside, preferably by an angle of approximately 90°. Thus, the tensioning devices may be arranged substantially below the support surface and in a space-saving manner on or in the frame.

In particular if the straps are substantially, i.e. in the scope of the forces usual with the use given, inelastic it is favorable if the tensioning devices comprise a resilient suspension, wherein the spring force exerted by the respective resilient suspension corresponds substantially to the pretensioning of the associated strap. This means that the tensioning devices are advantageously elastic themselves and may particularly have a smaller spring constant than the straps. Expediently, the adjustment of the pretensioning may thus be made by the variation of the spring deflection. The resilient suspension may be a resilient member, for instance, a coil spring, a leaf spring, a suspension by means of elastic materials (e.g. rubber, silicone), or a gas pressure spring, wherein here, too, particular importance should be attached to a preferably low noise development.

In connection with the resilient suspension it is of advantage if the maximum lift of the resilient suspension and/or the maximum spring deflection is preferably restricted at a value of ≦10 cm. Thus, in the case of typically temporary higher loads—for instance during propping with the elbow or during getting up—it is possible to avoid excessive or undesirably deep local sinking and/or yielding of the support surface, so that the corresponding activity is not or as little as possible impeded and/or hindered.

A particularly quick adjustment of the support apparatus is possible if the tensioning devices comprise a drive for adjusting the pretensioning. The drive may generally replace a manual adjustment and enables, for instance, also those users to adapt the pretensioning to their own demands who would have to resort to foreign help otherwise.

If the moment applied by the drive is sufficient to adjust the pretensioning during the use of the support apparatus, for instance, is 1.5 Nm per strap, depending on and/or adaptable to the weight class of the user and the strap breadth, the apparatus need not be relieved and/or left for and during the adaptation of the hardness. This is of advantage in particular in the case of frequent new adjustments and in the case of users with reduced mobility (particular cases of illness) of the support apparatus, who can thus take particular profit from the advantages of the apparatus.

A preferred variant of the connection between the strap and the drive is that the drive is connected to the strap via a leadscrew. The leadscrew offers high power gear ratio from a rotating into a translatory movement which enables, for instance, the use of electric motors, e.g. step motors, which are relatively weak as compared to the pretensioning. At the same time it is possible to achieve a high precision of the pretensioning adjustment due to the gear ratio.

If both a resilient suspension and a drive are used, it has turned out advantageous if the drive is operably arranged between the resilient suspension and the strap. In particular, in the case of this arrangement the drive is mounted to be dampened relative to the frame, so that possible vibrations caused by the drive are decoupled from the frame and noise is thus reduced. Irrespective of this, the connection between the drive and the strap is simpler if the resilient suspension is not mounted therebetween which could disadvantageously also absorb a torque.

A particularly comfortable adjustment of the support apparatus can be achieved in that the drives of the tensioning devices are controlled and/or are controllable separately, wherein a locally adjustable pretensioning of the straps can be adjusted in accordance with a pretensioning profile, e.g. automatically. In connection with an appropriate control it is thus possible to adjust different pretensioning profiles depending on the user and/or the momentarily preferred positioning, the position of the body or therapeutic needs.

Long-term modifications or particular time-dependent functions may further be implemented in that the drives of the tensioning devices are time-controlled and the pretensioning of the straps can be modified automatically as a function of time. Examples of use illustrating the advantage of such time-controlled modifications are the continuous redistribution of stressed parts of the body of patients or the adaptation to a user's phase of sleep. By means of an appropriate control program it is also possible to achieve specific medical effects such as pressure migrating systematically from the bottom (condal) or upwards (toward the heart) (alternating pressure), or support during the removal of tissue fluid (edemas).

In particular for observation, but also for the automated determination of the pretensioning adjustment it is helpful if the tensioning devices comprise a force sensor for determining the support pressure, wherein the force sensor is preferably firmly connected to the strap. The evaluation of the values transmitted by the force sensor allows for conclusions with respect to the position, the weight and possible pressure points and enables the automatic or semi-automatic adaptation of the pretensioning profile to the current use. If the force sensor is directly connected to the strap, it is possible to detect the force directly with utmost exactness and dynamics. For minor demands it is also possible to calculate the respective force indirectly via the control performance of the drive as measured.

A particularly reliable adjustment of the pretensioning profiles exists if the drives of the tensioning devices are controlled as a function of the support pressure determined by the force sensor. Under these circumstances it is, for instance, possible to react automatically to changes of the position of the user's body.

In order to ensure high safety for the users even when individual straps are loaded, the straps and/or the tensioning devices should have a tensile load which is at least by a factor 3 higher than the required strap pretensioning (depending on the user's weight class).

Apart from the excellent air permeability and the associated natural temperature control of the support surface the support apparatus according to the invention enables, due to the accessibility from the underside and the small thickness and compressibility of the straps forming the support surface, the use of a therapeutic equipment arranged at the underside, i.e. as an equipment for treatment in accordance with the above-described module structure, wherein the therapeutic equipment preferably comprises exchangeable and/or automatically switchable massage heads and/or treatment heads (local cooling or heating) as an equipment for massage.

A massage adapted to the anatomic conditions can be enabled in that the equipment for massage performs an automatic determination of the position of the user being present on the support surface by means of contact-based scanning and/or contactless, in particular capacitive, measurement. Thus, it is possible to determine the parts or regions of the body to be massaged irrespective of an absolute positioning. The values determined by the force sensors may possibly also be used for coarse orientation and/or initialization.

Since the temperature at the support surface is a central parameter for the comfort of the support apparatus, a heating, for instance, an electrical heating or an infrared radiator, and/or a cooling, for instance, a ventilator, a cooling head or a temperature control circuit with a temperature control medium, may advantageously be arranged at the head or foot ends of the bed as required, so that, depending on the environment and body temperatures, a comfortable temperature control of the support surface can be performed. In this context it is also desired to apply particular temperature profiles at the support surface.

For the use in the field of care or in hospitals it is desirable if the straps are manufactured of a heat, UV and moisture-resistant material and can be sanitized thermally and chemically. Such an easy-care apparatus which can, for instance, be sanitized easily with a hot water high pressure cleaner may also be offered under hygienically challenging conditions without restriction since a thick protective layer inserted between the support surface and the user is thus not required. Such protective layers, as they are, for instance, usual and necessary for the protection of mattresses, would strongly impair the air and temperature permeability of the apparatus as well as the possibilities of therapy/massage.

In order to largely avoid noise development and possible undesired shifting during the adjustment of the tensioning devices it is of advantage if the straps have a low-friction and/or slidable surface, wherein the friction resistance between the straps is so low that it is negligible in relation with the pretensioning, wherein any noise during the shifting of the straps is also minimized.

When used for immobile patients or for patients who are difficult to put into another bed in hospitals and/or in the field of care it is further beneficial if the support surface has an absorbability for X-rays which is comparable with a mattress or lower since it is thus possible to perform diverse methods of diagnosis directly on the support apparatus, which is particularly favored by the accessibility of the support surface from the underside.

The previously mentioned advantages may, for instance, be achieved if the straps consist substantially of polyester mesh work, artificial silk, Goretex or aramide ribbons, alternatively of hybrid fabrics (e.g. reinforced with carbon fibers). For increasing slidability, the straps may additionally have a coating, in particular a coating with Teflon.

If the frame is divided in longitudinal direction into preferably at least two, in particular four, partial segments interconnected by hinges, wherein the hinge axes are arranged transversely to the longitudinal direction of the frame, the support apparatus can support a plurality of body positions. The partial segments may be oriented in parallel or may be adjusted in their angular position mechanically and/or via auxiliary drives, for instance, commercially available linear drives, so-called lifting columns, which are frequently used for hospital beds. The same apparatus may, depending on the arrangement of the partial segments, either be used as an equipment for lying on or for sitting on, for lifting the head portion or the knee region.

For supporting the adjustment of the angular position of the partial segments, drives causing a particular adjustment and/or supporting a movement of the partial segments may be connected to the frame. The control of the position of the individual segments of the support and the presetting of different therapeutic massage programs and/or cooling or heating measures is performed via a human machine interface (abbreviated as HMI) or in a remote-controlled manner. Different security levels are provided for the change or input of the parameters. Therapists or physicians have, with a specific password, access to the highest security level, with the possibility of specifying all available parameters and programs for the user. Medical care personnel has access to a reduced field of parameters which is also protected by a specific password and/or may cause the initiation of predefined therapeutic treatment programs in a time-controlled manner. Functions released from higher security levels may be activated by the user at any time without a password. When using one bed for a plurality of users, user-specific characteristic data, for instance the stiffness profile of the straps, may be stored and retrieved in the HMI.

In particular in publicly accessible facilities, such as hospitals, nursing homes or hotels, where a support apparatus is used at different places and for different users, it is beneficial if the substructure has braked rolls for moving the apparatus as well as a preferably electrical, alternatively hydraulic or pneumatic auxiliary drive for lifting and lowering and/or inclining the support apparatus, in particular individual partial segments of the frame.

Likewise, in particular if a plurality of support apparatuses according to the invention are used in one single room, it is of advantage if the noise development is reduced to a minimum with all applications. Special emphasis will have to be put on the rolling guides, the resilient suspensions and the drives, but also the friction between the straps should be kept as low as possible. When using an equipment for massage or a cooling or heating device, a low noise development is also desired and can typically be achieved by suitable drives, such as direct drives with a toothed belt.

### SHORT DESCRIPTION OF THE DRAWINGS

The invention will be described in more detail in the following by means of particularly preferred embodiments, which it is not intended to be restricted to, though, and with reference to the drawings. The drawings show in detail:

FIG. 1 illustrates a schematic plan view of a support apparatus in accordance with the invention;

FIG. 2 illustrates a schematic detailed view of a rolling guide according to section II in FIG. 1;

FIG. 3 illustrates in a schematic plan view in partial FIG. 3A straps connected to tensioning devices at one side and in partial FIG. 3B straps connected to tensioning devices at both sides;

FIG. 4 illustrates a schematic vertical section through a tensioning device pursuant to FIG. 2;

FIG. 5 illustrates schematically an electronic control circuit for applying a pretensioning profile;

FIG. 6A illustrates a figurative view of a support apparatus (without strap mesh work) consisting of three modules with an equipment for massage;

FIG. 6B illustrates a schematic explosive view of the three modules pursuant to FIG. 6A;

FIG. 6C illustrates a perspective view of a massage head of the equipment for massage pursuant to FIG. 6A;

FIG. 6D illustrates a perspective view of a locally effective heating or cooling head pursuant to FIG. 6A; and

FIG. 7 with partial Figures A to F schematically illustrates six positions of the support apparatus pursuant to FIG. 6A.

### DETAILED DESCRIPTION OF THE DRAWINGS

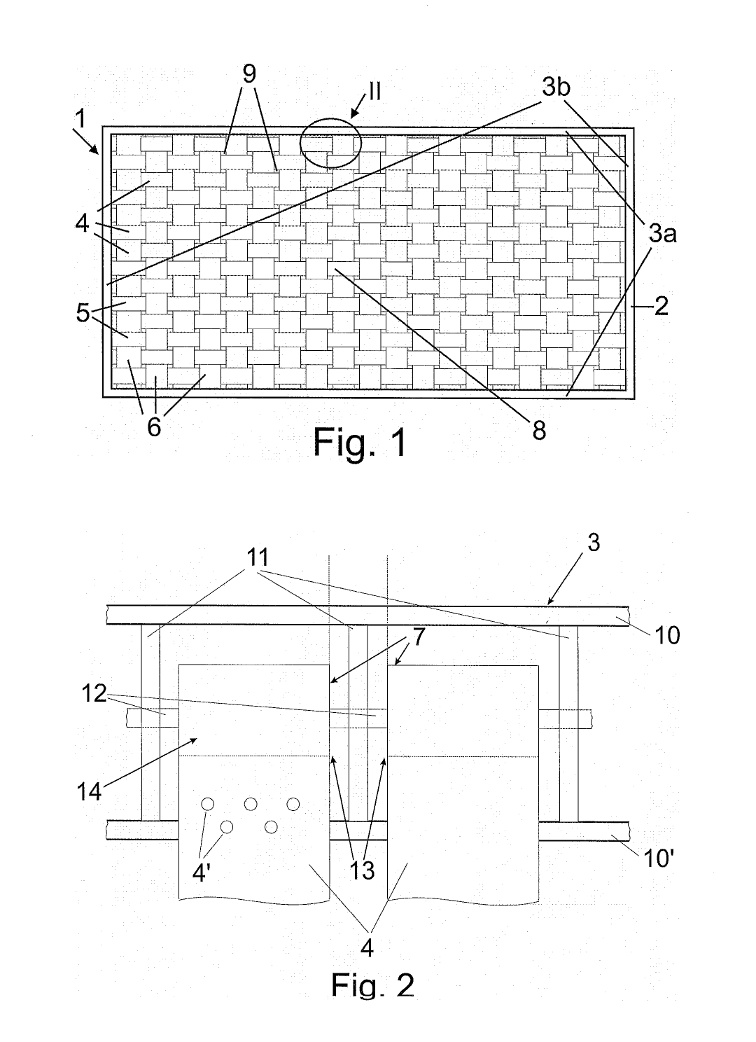

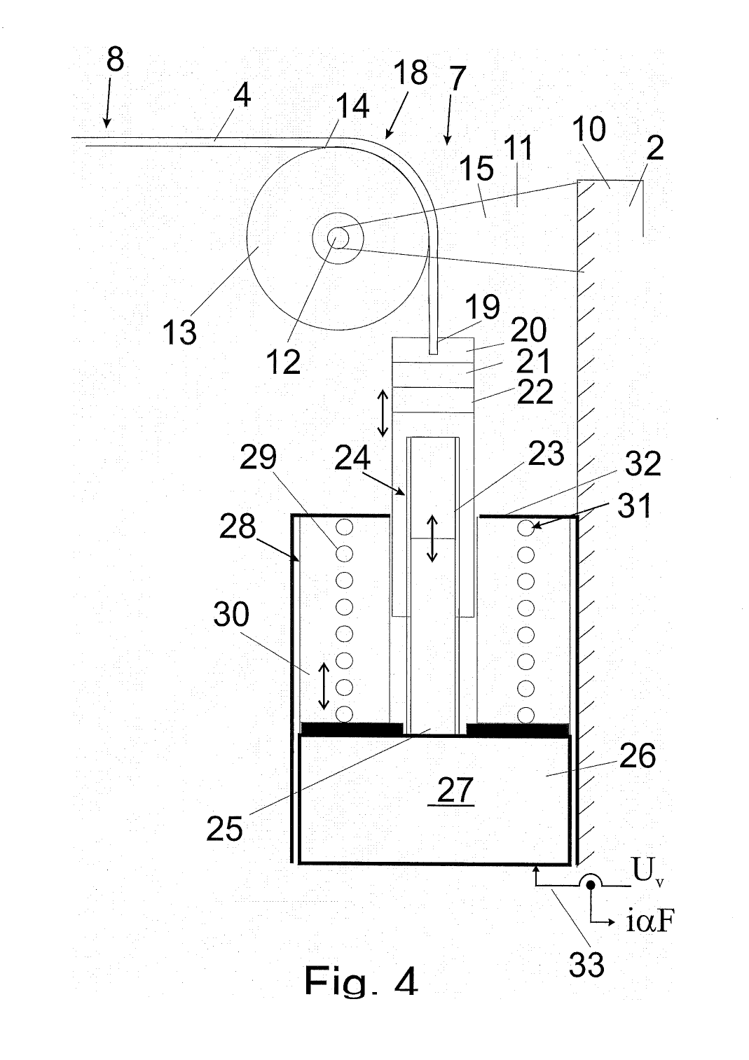

FIG. 1 illustrates in a schematic plan view a support apparatus 1 for an equipment for lying on, in particular a bed. The support apparatus 1 comprises a frame 2 rectangular in plan view. Straps 4 are tensioned in the frame 2 between opposite frame elements 3a, 3b. The straps 4 run in two groups in parallel either in the longitudinal direction of the frame 2 or transversely thereto and are arranged at equal distances. The distance, i.e. the free space 9 between the straps 4, should not exceed 10 mm so as to prevent breaking through, for instance, with the elbow, during propping. In a preferred embodiment the strap distance is approximately 5 mm. In order to avoid a slipping of the straps 4 transversely to the tensioning direction, the straps 4 are intertwined, wherein, for instance, a longitudinal strap 5 is alternatingly arranged above and below successive transverse straps 6. Depending on the strap breadth selected, alternative mesh work combinations (e.g. two respective longitudinal and/or transverse straps) may, however, also be chosen. The breadth of parallel straps 4 is substantially constant, wherein in the illustrated example the longitudinal straps 5 are narrower than the transverse straps 6. A strap breadth of at least 5 mm, preferably between 30 and 200 mm, in particular 50 mm, is a suitable compromise between local adjustability (comfort), strength, and economy. Narrower straps 4 enable a finer adjustment of the local stiffness and hence higher comfort by a more equal pressure distribution. They are of advantage in particular in the regions of high support pressures (sacrum, knee, shoulder and heel regions). Broader straps 4 require a less high technical and hence financial effort. The straps 4 are connected at both ends with the frame elements 3a, 3b, wherein the connection is made at least at one end and at least at one transverse side of the frame 2 via tensioning devices 7 (cf. FIG. 4). The face enclosed by the frame 2 is covered by the strap mesh work, so that the straps 4 form a support surface 8. The rectangular openings 9 remaining between the straps in the support surface 8 have edge lengths corresponding to the distances between the straps 4 and determine a relatively high air permeability of the apparatus. Additionally, perforations 4′ (cf. FIG. 2) (matrix of holes) may be provided in the straps 4 so as to further improve the ventilation of the support surface 8 from the underside.

As far as the material and the structure of the straps 4 are concerned, they need not only meet the mechanical requirements, for instance, a particular tensile strength, but they often have to meet additional demands with respect to hygiene and cleaning possibilities. It is particularly desired that plastics inserts—as they are necessary with usual mattresses or foam inserts—need not be used, which would strongly reduce comfort. If the straps 4 consist, for instance, of polyester mesh work, artificial silk, Goretex or aramide ribbons, alternatively of hybrid fabrics (e.g. reinforced with carbon fibers, without or with coating for increasing slidability, for instance, with Teflon), the support surface 8 can be cleaned directly with a high pressure cleaner and can be prepared for use in accordance with the hygienic demands, in particular can be sanitized.

FIG. 2 illustrates two tensioning devices 7 in accordance with the detail II in FIG. 1. It shows that the frame element 3a and/or 3b is constructed of two parallel longitudinal struts 10, 10′ with roll supports 11 arranged transversely therebetween. The strap breadth and the strap distance are not illustrated to scale, but would preferably be 50 mm and 5 mm, respectively. In parallel to the longitudinal struts 10, roll axes 12 are arranged in the roll supports 11, said roll axes serving to support the rolls 13 forming a rolling guide. The distance between the longitudinal struts 10, 10′ is larger than the diameter of the rolls 13, so that the distance between the roll 13 and the outer longitudinal strut 10 corresponds at least to the strength of the strap 4. Preferably, the height of the inner longitudinal strut 10′ is chosen such that the summit 14 of the roll 13 projects over the inner longitudinal strut 10′, so that the strap 4 is tensioned across it without touching it and/or rubbing on it. Alternatively, an inner longitudinal strut 10′ may also be omitted completely (cf. FIG. 4), wherein the roll axes 12 and thus the rolls 13 are then carried for example by freely protruding brackets 15 as roll supports 11.

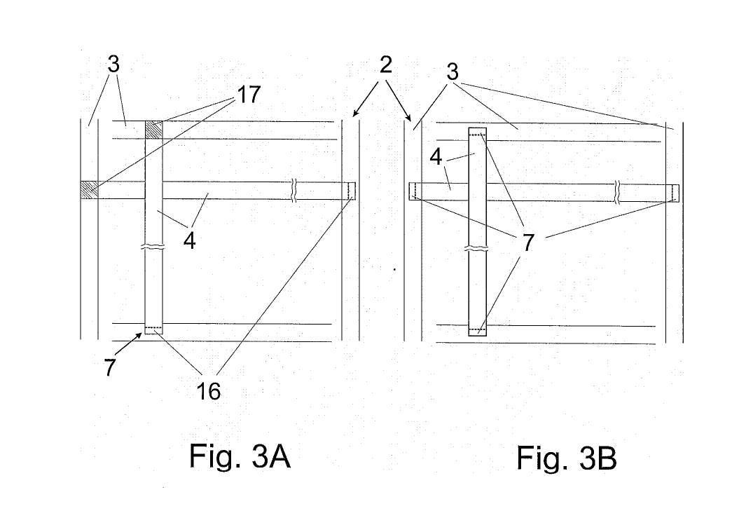

As is illustrated in FIG. 3A, the straps 4 may be connected to a tensioning device 7 at one end 16 only. The end 17 opposite to the tensioning device 7 is then, for instance, firmly connected to the frame 2. The tensioning devices 7 of all straps 4 may be arranged at one frame element 3a, 3b, or the tensioning devices 7 of successive parallel straps 4 are alternatingly arranged at the one and at the opposite frame element 3. The least manufacturing effort is achieved if all tensioning devices 7 are arranged at only two frame elements 3 (one transverse element and one longitudinal element) positioned at right angles to one another.

Higher comfort is, however, provided by tensioning devices 7 which are connected at both sides with the straps 4, as is illustrated in FIG. 3B. Due to the lower friction between the straps 4 this variant also enables a reduction of the noise development during use.

FIG. 4 schematically illustrates the construction of a preferred embodiment of a tensioning device 7 in detail. As already explained in connection with FIG. 2, the strap 4 is guided via a rolling guide 18 with a roll 13. As may be seen in detail, the rolling guide 18 guides the strap 4 starting out from a horizontal support surface 8 along a quarter segment of the roll 13 by 90° in a vertical. The roll 13 of the rolling guide 18 is mounted to be rotated about a roll axis 12, wherein the roll axis 12 is arranged in the bracket 15 provided as a roll support 11 which is in turn connected to the outer frame element 10. The strap end 19 pointing vertically downward is arranged and retained in a clamp 20. The clamp 20 is connected to a force sensor 22, e.g. with a strain gauge element, via a bearing 21. At the side of the force sensor 22 opposite to the bearing 21 the force sensor is coupled with a leadscrew 24 comprising a fine thread 23 and is secured against twisting. The force sensor 22 thus measures the traction directly between the leadscrew 24 and the strap 4. The leadscrew 24 comprising the fine thread 23 is connected to the shaft 25 of a drive 26, in particular an electric motor 27. The drive 26 itself is finally connected to the frame 2 of the support apparatus via a resilient suspension 28. The resilient suspension 28 is formed by a pressure spring 29 and/or an additionally resilient filler 30, for instance, rubber or silicone, and is compressed by the strap pretensioning. In the illustrated example the strap tensioning is predetermined by the position of the shaft 25 in the leadscrew 24 which is adjustable by means of the drive 26. The resilient suspension 28 is supported via the horizontal contact surface 32 which is firmly connected to the frame element 10. As an alternative embodiment a joint drive for a frame side for a frame element 3a and/or 3b could also be chosen, wherein the adjustment of the respective strap pretensioning is performed by a specific coupling of the drive (e.g. movable gear wheel) to the leadscrew 24.

The maximum lift of the resilient suspension 28 is defined by the maximum compressibility of the spring elements 29 and/or 30. In the case of a—practically impossible—complete compression of the resilient suspension 28 the drive 26 would yield in upward direction up to an abutment at the contact surface 32 maximally. The adjustment of the mechanical strap pretensioning is performed via the supply line 33 of the drive 26, wherein an appropriately poled current or in the case of a step motor a pulse sequence over a particular period causes the desired leadscrew rotation in the one or the other direction. If the strap 4 does not act as a suspension itself, the translatory movement caused by the leadscrew 24 results in a compression or decompression of the spring 29 and/or of the resilient filler 30 and thus in an increase or decrease of the pretensioning of the strap 4. Due to the deflection of the strap 4, the force corresponding to the pretensioning acts, without the loading of the support surface 8, not only horizontally between the opposite frame elements 3a and/or 3b, but also vertically between the rolling guide 18 and the contact surface 32 of the resilient suspension 28.

The lift of the leadscrew 24 is expediently at most equal to the maximum lift of the resilient suspension 28, wherein the length of the vertical strap section, in particular in the case of low pretensioning, has to be selected such there is no collision of the strap clamp 20 and the roll 13 when the support surface is loaded.

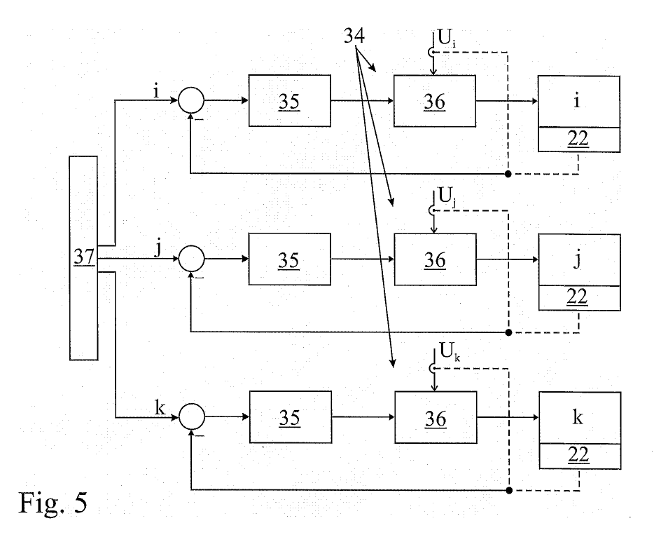

The substantially rigid connection between the drive 26 and the force sensor 22, in particular without interposed suspension, as illustrated in FIG. 4 achieves a direct and undisturbed mechanical feedback from the drive 26 to the force sensor 22 in a control loop 34, as is, for instance, illustrated in FIG. 5. Here, control loops 34 for three independent tensioning devices 7 are illustrated schematically, which obtain their respective nominal values to which the strap pretensioning is set, from a joint pretensioning profile. The individual control loops 34 comprise, in addition to the force measurements and/or force sensors 22, also controllers 35 and actors 36 (corresponding to the drives 26). In practice, the controllers receive a selectable pretensioning profile via a central programmed specification of the nominal value by the HMI 43 through the input 37. The controllers 35 supply the required control signal for the actors 36 on the basis of the actual value detection by the force measurement 22 and/or the current measurement (Uᵢ, Uⱼ, Uₖ, etc.).

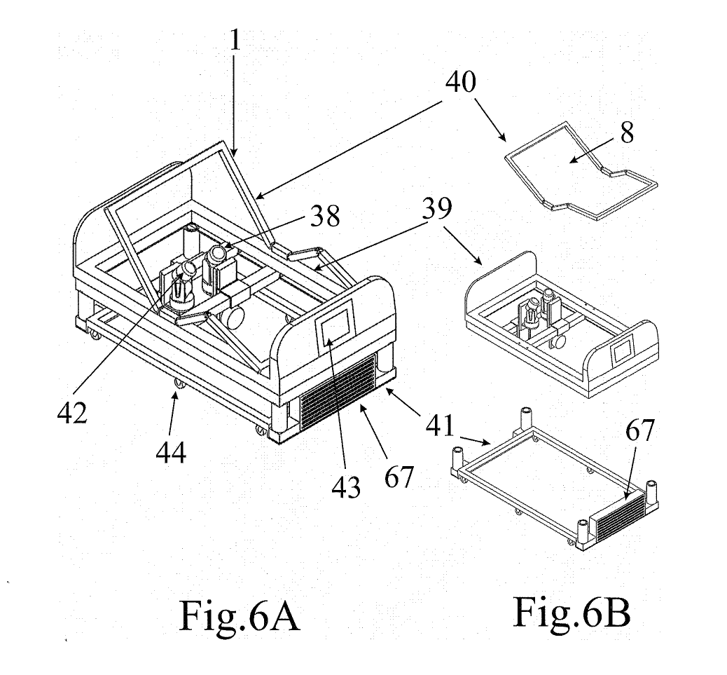

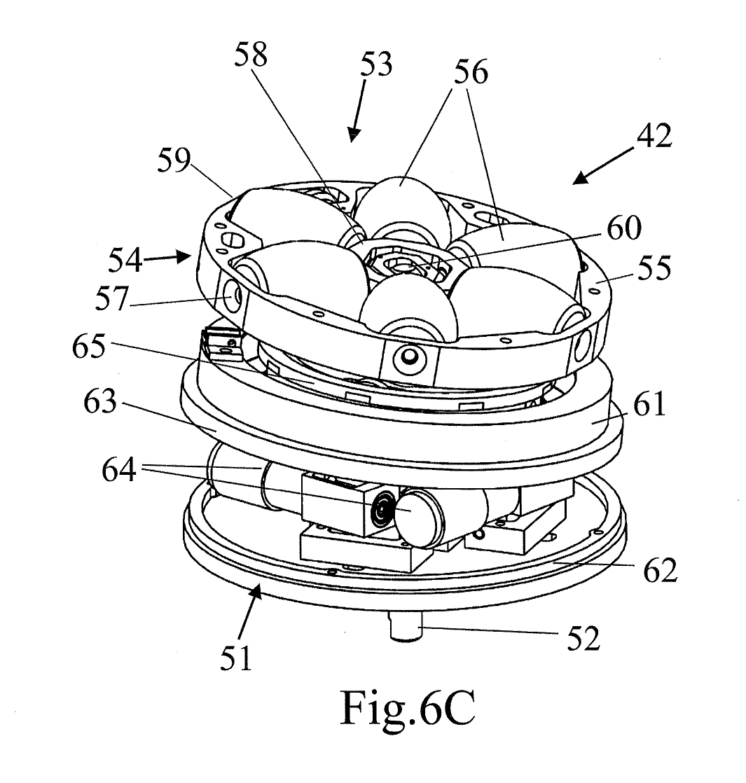

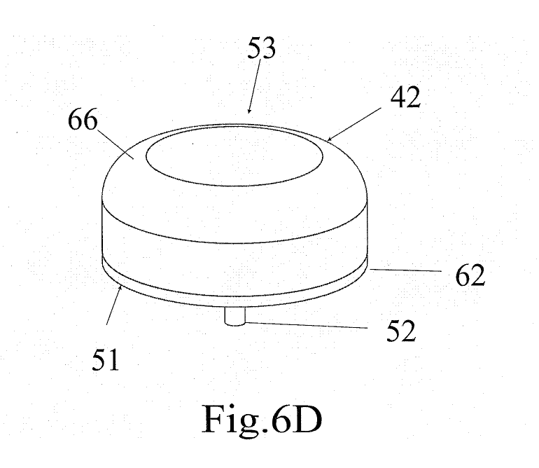

A central advantage of the present support apparatus 1—apart from the gain of comfort—is the accessibility of the support surface 8 also from the underside and the application scenarios enabled by that. The modular structure of the support apparatus 1 illustrated by way of example in FIGS. 6A and 6B is particularly beneficial for the versatility of applications. Even if merely an equipment for massage 38 is illustrated here as an equipment for treatment 39, it is understood by a person skilled in the art that the most varied equipments for treatment 39 can be used between the first module 40 (illustrated only schematically, without straps, in FIG. 6) with the support surface 8 and the third module 41 which forms a universal substructure. In particular, reference is made here to the use of a “heating tool” or a “cooling tool” (cf. FIG. 6D), for instance, an infrared radiator and/or an electrical resistance heating or a precooled cooling head—to be used optionally instead of a massage head 42—in the equipment for massage 38 for the local heating or cooling of the support surface 8 and/or of regions of the user's body. For a global heating or cooling of the support surface 8 the third module 41 may comprise, for example at the foot end, a heating (e.g. an electrical heating or an infrared radiator) and/or a cooling aggregate 67 (e.g. with Peltier elements or specific cooling agents and cooling circuits) along with a ventilation with controllable speed.

Due to the small thickness of the support and/or of the straps 4, good access can be achieved if an equipment for massage 38 is used, which is particularly beneficial for local treatments. Consequently, massage movements controlled pursuant to therapeutic principles can be performed from the underside of the support apparatus 1 for the neck, the back and the leg region, such as effleurage, tumbling, knocking and vibrating. For performing the different techniques, the equipment for massage 38 comprises massage heads 42 (cf. FIG. 6C) which can be moved, for instance, with six degrees of freedom (three rotation axes and three translation directions). Moreover, different massage heads 42 may be provided which are selected and used manually or automatically. Possible applications are, for instance, lymphatic drainage, (micro) stimulations for avoiding damage caused to health by long immobilization (e.g. decubitus) and loss of feeling for individual parts of the body (in particular shoulder, sacrum region and heels), psychical relaxation, the strengthening of the immune system, the improvement of micro circulation and the prevention of pressure ulcers. The automated performance of appropriate therapies does not only enable relief of the nursing staff, but favors also a quick recovery and hence a shorter stay especially in hospitals. For preparation of the respective massage program the current position of the person to be massaged is determined at the beginning, but also during the massage by an extensive, very gentle scanning, corresponding to effleurage, with the equipment for massage 38, or contactless (e.g. with inductive, optical or infrared, preferably capacitive, sensors).

FIG. 6C illustrates a massage head 42 in detail. The massage head 42 is connected to the equipment for massage 38 via the underside 51 and via an axis of rotation 52 (cf. FIG. 6A). At the upper side 53 the massage head 42 comprises a circular massage tool 54 with a bearing crown 55 and six bellied massage rollers 56 mounted to rotate therein. The axes 57 of the massage rollers 56 form the spokes of the bearing crown 55 and are arranged radially between a central hub 58 and an outer ring 59. The massage tool 54 is not only rotatable about the axis of rotation 52, but due to a gimbal suspension 60 of the hub 58 also pivotable in any direction relative to the rest of the massage head 42. Below the massage tool 54 the massage head 42 comprises a rotating guide 61 of the bearing crown 55. An elastic sealing (not illustrated) is provided between a limiting ring 63 and the circular bottom 62 of the massage head 42. An elastic sealing is also provided for the upper region of the massage head 42. Mounting is performed on the intermediate ring 65 and the rotating axis 52 by means of a sealing ring. A plurality of drives 64 can be seen above the bottom 62. The limiting ring 63 limits the pivot angle of the massage tool 54 and thus protects the drives 64 from collisions with the massage tool 54. The drives 64 may determine the rotation of the massage head 42, on the one hand, and/or the rotation and pivoting of the massage tool 54.

In the case of sleep disorders or for gain of comfort, an equipment for massage 38 may execute time-controlled massage programs for supporting the process of falling asleep, for shortening wake periods or as a wake-up function. In the case of an automation the massage is available at any time and several times a day, which is hardly possible with manual massages performed by nursing personnel. Since the described support apparatus 1 along with the tensioning devices 7 and the equipment for massage 38 operates very silently, a massage, a local or global cooling or heating or a modification of the strap pretensioning need not be omitted even during quiet hours nor even in shared rooms.

As may be seen in FIG. 6A, the support apparatus 1 may also be connected to a HMI 43 which enables, for instance, the configuration of a pretensioning profile or of a massage program. The data of the force sensors 22 could also be recorded, evaluated and referred to, for instance, for selecting or optimizing the massage.

It is to be understood that other medical equipments and appliances such as, for instance, detectors or recording devices for an X-ray diagnosis, may also be used as an equipment for treatment 39.

FIG. 6D illustrates a massage head 42 for local heating or cooling. The massage head 42 is connected to the equipment for massage 38 at the underside 51 and via an axis of rotation 52 (cf. FIG. 6A). For local heating, an electrical resistance heating or heating lamp is integrated in the massage head housing 66 of the massage head. For local cooling, the massage head housing 66 is made of a material with high heat capacity, e.g. copper. For cooling, the massage head 42 is cooled to a predeterminable temperature in its tool holder.

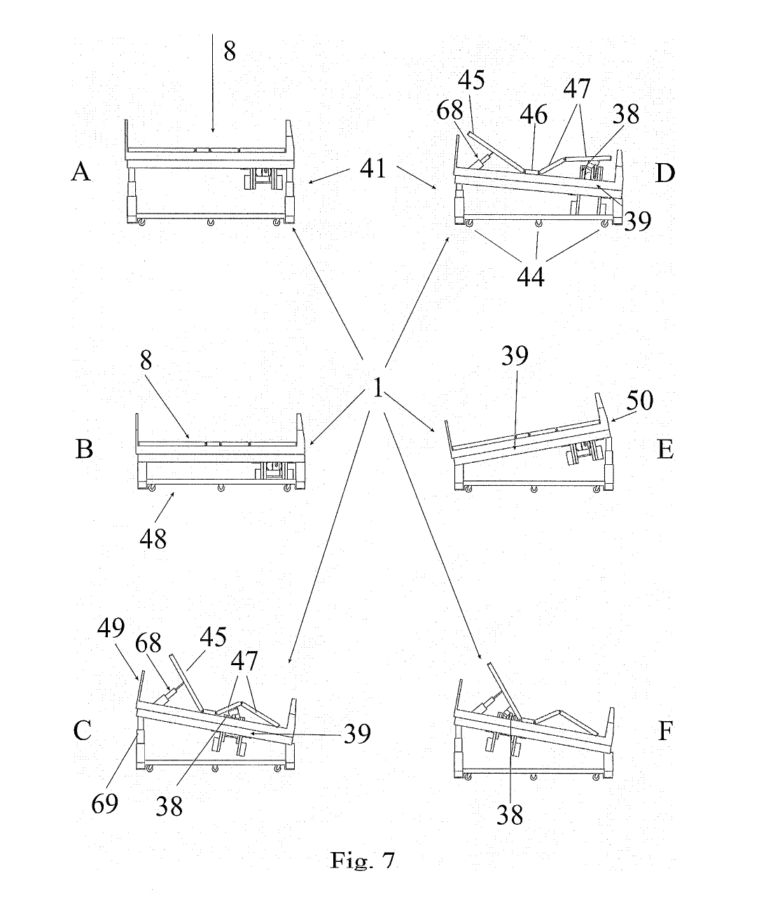

For using the support apparatus 1, for instance, as a sickbed, it is possible to implement specific functions in the third module 41—the substructure. As may be seen in FIG. 7, these include the mobility established by rolls 44, the fixing by means of brakes (not illustrated), but also the lifting, lowering and inclining of the entire support surface 8 or of partial segments 45, 46, 47 of the support surface 8 (cf. partial Figures A to F in FIG. 7) so as to facilitate the care work and/or as safety measures. The equipment for treatment 39 may follow the modifications of the support surface 8, as is illustrated here by way of example with the equipment for massage 38. The partial Figures illustrate in detail:

A A flat treatment position for any positions of the body, wherein the support surface 8 is advantageously arranged roughly at hip height and/or working height of the treating personnel.

B A flat entry position, wherein the distance of the support surface 8 from the floor 48 corresponds roughly to the sitting height of the user, so that easy entering and getting off is enabled. This position is also chosen as a specific sleeping position if there is the danger of falling out, or as an emergency position (for reanimation).

C An S-shaped sitting position, wherein a back segment 45 is tilted by approximately 45° (e.g. by means of a schematically drawn pressure cylinder 68), two leg segments 47 are arranged to support an angled leg position, and the entire second module 39—e.g. by means of a telescopic cylinder means 69—comprises an inclination ascending toward the head end 49. This position is also used as an emergency position (Trendelenburg position). It must be possible to adjust positions C and E particularly quickly. The equipment for massage 38 is arranged in a position for massage of the user's thighs.

D A slightly erected lying position with raised back and leg segments 45, 47.

E A flat lying position, wherein the second module 39 has an inclination ascending toward the foot end 50.

F A sitting position as in partial figure D, wherein the equipment for massage 38 is arranged in a position for massage of the user's lumbar vertebra region; wherein it is possible to adjust positions C and E particularly quickly.

Furthermore, an electrical auxiliary drive, for instance, may be provided to facilitate the change of location of beds that are moved frequently.

As mentioned, two lifting means 68, 69 are illustrated schematically by way of example in FIG. 7C. Comparable (or other known) lifting means and/or lifting columns may be used for the other segments and/or modules, which is not illustrated in the drawings for reasons of simplification.

Even if the illustrated support apparatus 1 can unfold its advantages especially as a sickbed in the field of hospitals, it may also be used in the working, private, care and wellness fields in the form of a bed or as a chair, seat (e.g. in transport means such as car, railway, bus or aircraft), as fauteuils or as a lounger (e.g. as a sun lounger in the outdoor area). In the private field the arrangement may possibly be appropriately simplified and a modular construction may, for instance, be omitted.

1-28. (canceled)

29. A support apparatus (1) for lying or sitting equipment, comprising an at least partially fixed frame (2) and comprising intertwined tensioned straps (4) which define a support surface (8) and are accessible from the upper support side of the support surface, wherein at least some of the straps (4) are connected to the frame (2) by means of separately adjustable tensioning devices (7) such that the pretensioning of the straps (4) is locally adjustable, wherein the tensioning devices (7) are arranged between the straps (4) and the frame (2) and that the straps (4) are also accessible from the underside of the support surface (8) such that the support surface (8) is accessible from the underside, that the tensioning devices (7) comprise a drive (26) for adjusting the pretensioning, and that the drives (27) of the tensioning devices (7) are controlled and/or controllable separately such that therewith the hardness of the support surface (8) is locally adaptable at any time, wherein a locally adjustable pretensioning of the straps (4) is adjustable in accordance with a pretensioning profile, e.g. automatically.

30. The support apparatus according to claim 29, wherein the frame (2) along with the straps (4) forms a first module (40) having an equipment for treatment which forms a second module arranged at the underside thereof, said equipment for treatment in turn being arranged on a third module formed by a substructure (41).

31. The support apparatus according to claim 29, wherein the straps (7) connected to a tensioning device (7) are firmly connected to the frame (2) at their end (17) opposite to the tensioning device.

32. The support apparatus according to claim 29, wherein the straps (4) connected to tensioning devices (7) are connected to a tensioning device (7) at both ends.

33. The support apparatus according to claim 29, wherein a maximum of ten straps (4), preferably one single strap (4), is connected to each tensioning device (7).

34. The support apparatus according to claim 29, wherein the tensioning devices (7) comprise a sliding or rolling guide (18) of the straps (4).

35. The support apparatus according to claim 34, wherein the sliding or rolling guide (18) deflects the straps (4) in the direction of the underside, preferably by an angle of approximately 90°.

36. The support apparatus according to claim 29, wherein the tensioning devices (7) comprise a resilient suspension (28), wherein the spring force exerted by the resilient suspension (28) substantially corresponds to the pretensioning of the associated strap (4).

37. The support apparatus according to claim 36, wherein the maximum lift of the resilient suspension (28) is restricted, preferably at a value of ≦10 cm.

38. The support apparatus according to claim 29, wherein the moment applied by the drive (26), preferably at least 1.5 Nm per strap (4), is sufficient for adjusting the pretensioning during the use of the support apparatus (1).

39. The support apparatus according to claim 29, wherein the drive (26) is connected to the strap (4) via a leadscrew (24).

40. The support apparatus according to claim 29, wherein the drive (26) is operably arranged between the resilient suspension (28) and the strap (4).

41. The support apparatus according to claim 29, wherein the drives (26) of the tensioning devices (7) are time-controlled and that the pretensioning of the straps (4) can be modified automatically as a function of time.

42. The support apparatus according to claim 29, wherein the tensioning devices (7) comprise a force sensor (22) for determining the support pressure, wherein the force sensor (22) is preferably firmly connected to the strap (4).

43. The support apparatus according to claim 42, wherein the drives (26) of the tensioning devices (7) are controlled as a function of the support pressure determined by the force sensor (22).

44. The support apparatus according to claim 29, wherein the tensile strength of the straps (4) and/or the tensioning device (7) can be adapted to the person using the support and comprises a tensile load which is at least by a factor three higher than the maximally occurring strap tensioning.

45. The support apparatus according to claim 30, wherein the equipment for treatment (39) comprises an equipment for massage (38), preferably an equipment for massage (38) with exchangeable or automatically switchable treatment heads (42), in particular for massage or for heat treatment.

46. The support apparatus according to claim 45, wherein the equipment for massage performs an automatic determination of the position of the user being present on the support surface by means of contact-based scanning and/or contactless, inductive, optical, thermal, in particular capacitive, measurement.

47. The support apparatus according to claim 29, wherein a heating, for instance, an electric heating or an infrared radiator, and/or a cooling, for instance, a ventilator or a temperature control circuit (67) with a temperature control medium, is arranged at the head or foot end of the bed (8).

48. The support apparatus according to claim 29, wherein the straps (4) are manufactured of a heat, UV and moisture-resistant material and are thermally and chemically sanitizable.

49. The support apparatus according to claim 29, wherein the straps (4) comprise a low-friction surface, wherein the friction resistance between the straps (4) is negligible in relation with the pretensioning.

50. The support apparatus according to claim 29, wherein the support surface (8) has an absorbability for X-rays which is comparable with a mattress or lower.

51. The support apparatus according to claim 29, wherein the straps (4) consist substantially of polyester mesh work, artificial silk or aramide ribbons, alternatively of hybrid fabrics, for instance, reinforced with carbon fibers, and preferably have a coating for increasing slidability, in particular a coating with Teflon.

52. The support apparatus according to claim 29, wherein the frame (2) is divided in longitudinal direction into preferably at least two, in particular four, partial segments (45) interconnected by hinges, wherein the hinge axes are arranged transversely to the longitudinal direction of the frame.

53. The support apparatus according to claim 29, wherein linear auxiliary drives, in particular lifting columns, for adjusting the angular position of the partial segments (45) are connected to the frame.

54. The support apparatus according to claim 30, wherein the substructure (4) comprises rolls with braking means (33) for moving and fixing the apparatus (1) as well as a preferably electrical, alternatively hydraulic or pneumatic, auxiliary drive for lifting and lowering and/or inclining the first and second modules (40, 39), in particular individual partial segments (45) of the frame (2).
